(19) United States
(12) Patent Application Publication
(10) Pub. No.: US 2026/0240917 A1
(43) Pub. Date: Aug. 20, 2026

(54) MEDICAL PRODUCTS MADE FROM WHARTON'S JELLY FOR PROMOTING BONE REGENERATION

(71) Applicant: NATIONAL YANG MING CHIAO TUNG UNIVERSITY, Taipei City (TW)

(72) Inventor: Yu-Show FU, Taipei City (TW)

(73) Assignee: NATIONAL YANG MING CHIAO TUNG UNIVERSITY, Taipei City (TW)

(21) Appl. No.: 18/843,544

(22) PCT Filed: Mar. 1, 2022
(86) PCT No.: PCT/CN2022/078588

§ 371 Date: Sep. 3, 2024

(51) Int. Cl. A61K 35/51; A61P 19/08

(52) U.S. Cl. CPC A61K 35/51; A61P 19/08

(57) ABSTRACT

The present invention relates to a medical product made from umbilical cord Wharton's jelly; wherein the medical product is obtained by either of two methods below: method (1): treating Wharton's jelly taken from an umbilical cord with a cryoprotective agent, followed by cryopreservation, and thawing before use; or method (2): lyophilizing the Wharton's jelly taken from an umbilical cord directly, and storing at room temperature in dry condition before use. The umbilical cord Wharton's jelly product is used for preparing a medicament for promoting osteogenesis, which can increase the expression of alkaline phosphatase in osteoblasts, increase the expression level of bone differentiation genes in osteoblasts, promote the differentiation of osteoblasts, and enhance the expression of calcium deposition, and can also effectively increase the production of bone, enable osteogenesis at bone defects, improve the active expression of osteoblasts at bone defects, or promote the development of new bone.

### FIELD OF THE INVENTION

The present invention pertains to a medical product made from Wharton's Jelly of umbilical cord with the effect of promoting bone regeneration.

### BACKGROUND OF THE INVENTION

Bones may be damaged or lost due to trauma, fracture, infection, or tumor metastasis. When bone defect is greater than twice the diameter of the diaphysis, the defect cannot naturally heal despite implanting various bone regenerative materials, known as critical-sized bone defect. Furthermore, if the fracture site is too large, the risks of subsequent fractures will increase significantly. In addition, the regeneration of compact bone defects is relatively difficult. Therefore, how to repair compact bone defects is a difficult problem in clinical orthopedics today.

Currently, common clinical bone materials are generally divided into three types. The first type is autologous bone graft whereby bone tissues are surgically obtained from other body parts and used to fill up the bone defect region. The drawbacks of this method include a long recovery period and the inability to extract bone tissue with matching area. The second type is allogeneic bone graft, i.e., excessive bone tissue removed from other patients who required surgical treatment is used after post-processing treatment. In addition to ethical issues, the risks of immune infection are present. Thus, the researchers in this field aim to develop suitable medical materials in an effort to overcome challenges in clinical treatment.

### SUMMARY OF THE INVENTION

The present invention provides a medical product made from Wharton's Jelly of an umbilical cord, which has an effect of promoting bone regeneration, wherein the medical product is made by either of two methods below: method (1): treating Wharton's Jelly taken from a maternal umbilical cord with a cryoprotective agent, followed by cryopreservation, and thawing before use; and method (2): lyophilizing directly the Wharton's Jelly taken from the maternal umbilical cord, and storing at room temperature in dry condition before use.

According to the present invention, the medical product made from Wharton's Jelly is obtained by the method (1), which comprises the steps of treating it with a cryoprotective agent and then freezing, and keeping the product in a frozen status before use, which is referred to as WJF (Wharton's Jelly Frozen) and still retains the cell bodies.

According to an embodiment of the present invention, the cryoprotective agent is a solution containing 10% dimethyl sulfoxide (DMSO).

According to an embodiment of the present invention, the cryopreservation is performed in liquid nitrogen.

In a particular example of the present invention, the medical product made from Wharton's Jelly is first protected with a cryoprotective agent (e.g., 10% DMSO solution), then frozen for preservation, and thawed before use.

According to the present invention, the medical product made from Wharton's Jelly is obtained by the method (2), which comprises the steps of taking a piece of Wharton's Jelly from maternal umbilical cords, freeze-drying it directly, and keeping it at room temperature in dry conditions before use, which is referred to as WJD (Wharton's JellyDried) and contains no cell bodies.

According to an embodiment of the present invention, the frozen product is stored at −80° C.

In another particular example of the present invention, the medical product made from Wharton's Jelly is obtained by the method comprising taking a piece of fresh Wharton's Jelly, freezing it directly at −80° C., and then freeze-drying it to drain the moisture from the tissues.

According to the present invention, the medical products made from Wharton's Jelly obtained by the two methods above can increase performance of alkaline phosphatase in osteoblasts, increase level of expression of bone differentiation genes in the osteoblasts, promote differentiation of the osteoblasts, enhance calcium deposition, effectively enhance production of bone, enable osteogenesis within a bone defect, enhance activity of the osteoblasts within the bone defect, or induce development of newly formed bone.

In one aspect, the present invention provides a use of the medical products made from Wharton's Jelly in preparation of a medicament for promoting bone regeneration.

The details of one or more embodiments of the invention are set forth in the description below. Other features or advantages of the present invention will be apparent from the following detailed descriptions of several embodiments, and also from the appending claims.

### BRIEF DESCRIPTION OF THE SEVERAL VIEWS OF THE DRAWINGS

The foregoing summary, as well as the following detailed description of the invention, will be better understood when read in conjunction with the accompanying drawings. For the purpose of illustrating the invention, there are shown in the drawings embodiments which are presently preferred. It should be understood, however, that the invention is not limited to the precise arrangements and instrumentalities shown.

In the drawings:

FIG. 1. shows the histological and morphological analysis of freshly frozen, or freeze-dried Wharton's Jelly according to the present invention, in which FIG. 1A shows a freshly frozen Wharton's Jelly (WJF) taken from the umbilical cord; and FIG. 1B shows a freeze-dried Wharton's Jelly matrix (WJD), which is paraffin-embedded and sectioned. FIG. 1A1 and FIG. 1B1 are shown by H&E staining, the freshly WJF shows a dense structure and the tissue is rich in mesenchymal stem cells, while the WJD shows a sparse and porous structure. FIG. 1A2 and FIG. 1B2 are showed by Sirius Red stain, the collagen of freshly WJF is arranged in bundles and densely packed, whereas the collagen of WJD surrounds the periphery of the voids. FIG. 1A3 and FIG. 1B3 are showed by PAS stain, and polysaccharides fill the tissue of the Wharton's Jelly. FIG. 1C and FIG. 1D show the scanning electron microscopy of WJD, showing porous structures of different sizes. FIG. 1E shows the freeze-dried Wharton's Jelly. Whether it is paraffin-embedded sections quantified by HE staining, or analyzed by scanning electron microscopy, there are showed porous structures of varying sizes. Moreover, the distribution of pore diameters is mostly in the range of 10-70 micrometers.

FIG. 2. shows the co-culture of rat osteoblasts (ROB) with either of two Wharton's Jelly products enhances the expression level of bone differentiation genes in osteoblasts, in which FIG. 2A shows that ROB cultured in vitro alone or co-cultured non-directly with WJD or WJF for 14 days are subjected to RT-PCR assay for RUNX2 in rats, and the results show that all groups have the expression of rat RUNX2; and FIG. 2B shows that the real time RT-PCR quantifies the rat RUNX2 expression in ROB. The results show a significant increase in the rat RUNX2 expression level in osteoblasts of the ROB/WJD group and the ROB/WJF group, compared to the ROB group (p<0.05). *: Significant difference compared to ROB group, p<0.05.

FIG. 3. shows the quantification of alkaline phosphatase, showing co-culture with either WJD or WJF elevates the expression level of alkaline phosphatase in ROB, in which FIG. 3A is a graph showing non-direct and direct co-culture models of ROB with WJD or WJF; FIG. 3B shows cell staining for alkaline phosphatase after 14 days of culture; and FIG. 3C shows quantification of intracellular alkaline phosphatase concentration, and the level of alkaline phosphatase expressed in ROB from the ROB/WJD group, the ROB+WJD group, the ROB/WJF group, or the ROB+WJF group is significantly increased, compared to the ROB alone group, as shown by OD 595 absorbance values (p<0.05). *: Significantly different compared to the ROB group, p<0.05.

FIG. 4. shows that co-culture of ROB with either of two Wharton's Jelly products promotes the performance of osteoblast calcium deposition, in which FIG. 4A shows ROB cultured in vitro alone or co-cultured non-directly with WJD or WJF for 21 days is stained with alizarin red S, and the gradually magnified images show that all groups of osteoblasts exhibit calcium deposition after treatment with bone differentiation medium; and FIG. 4B shows quantification of the concentration of calcium ions in each group, and the OD 540 absorbance values show that both the calcium deposition contents in the ROB/WJD group and the ROB/WJF group are higher than that in the ROB group (p<0.05). *: Significant difference compared to ROB group, p<0.05.

FIG. 5. shows that the implantation of either of two Wharton's Jelly products in the treatment group effectively promotes osteogenesis in rats with calvarial defects, as observed via micro-computed tomography scan (micro-CT). Bone healing in rats with cranial defects is tracked via micro-CT at the first, second, third, fourth, and fifth months after surgery, in which FIG. 5A shows that in the Injury+Saline group, there is only a small amount of bone growth along the periphery of the defect, while in the Injury+WJD and Injury+WJF groups, there is free new bone not only around the periphery of the defect, but also within the defect area, and FIG. 5B shows the boundary of bone defect in red dashed line to quantify the percentage of new bone area within the defect area. Results show that the percentage of new bone area is significantly increased in the Injury+WJD and Injury+WJF groups, compared to the Injury+Saline group. *: Significantly different compared to the Injury+Saline group at the same month of age, p<0.05.

FIG. 6. shows that the percentage of new bone area in rats with calvarial defects is effectively increased by either of two Wharton's Jelly products in the treatment groups, as observed by H&E staining, in which FIG. 6A shows low magnification panoramic images of horizontal skull tissue sections stained by H&E staining, and the area of the bone defect is marked by white dotted line; FIG. 6B shows the border areas of the bone defects gradually enlarged from low magnification, respectively; FIG. 6C shows the inner area of the bone defect (The scale bars are 1 mm and 200 μm, respectively) gradually enlarged from low magnification; and FIG. 6D shows percentage of new bone area is quantified by H&E staining. The results show that the percentages of new bone areas in the Injury+WJD and Injury+WJF groups are significantly increased, compared to the Injury+Saline group (p<0.05, n=5). *: Significant difference compared to Injury+Saline group, p<0.05.

FIG. 7. shows the implantation of either of two Wharton's Jelly products effectively increases the distribution of mature osteoblasts in rats with calvarial defects, as observed by ALP staining, in which FIG. 7A shows low magnification panoramic images of horizontal skull tissue sections stained with ALP in each group; FIG. 7B shows the border areas of the bone defects gradually enlarged from low magnification, respectively; FIG. 7C shows the inner area of the bone defects; and FIG. 7D shows percentage of mature osteoblast area quantified by ALP staining. The results show that the percentages of distribution areas of mature osteoblasts in the Injury+WJD and Injury+WJF groups are significantly increased, compared to the Injury+Saline group (p<0.05, n=5). *: Significant difference compared to Injury+Saline group, p<0.05.

FIG. 8. shows the implantation of either of two Wharton's Jelly products effectively increases the distribution of immature osteoblasts in rats with cranial defects as observed by immunohistological staining with anti-osteocalcin, in which FIG. 8A shows low magnification panoramic images of horizontal skull tissue sections stained with anti-osteocalcin in each group; FIG. 8B shows the border areas of the bone defects gradually enlarged from low magnification, respectively;

FIG. 8C shows the inner areas of the bone defects; and FIG. 8D shows percentage of immature osteoblast area is quantified by anti-osteocalcin staining. The results show that the percentages of distribution areas of immature osteoblasts in the Injury+WJD and Injury+WJF groups are significantly increased, compared to the Injury+Saline group (p<0.05, n=5). *: Significant difference compared to Injury+Saline group, p<0.05.

FIG. 9. shows the trend of bone development in the newborn skull. Immunohistological staining with anti-osteocalcin is used to identify immature osteoblasts, ALP tissue staining is used to identify mature osteoblasts, and H&E staining is used to identify mature bone. The quantitative data from the three types of staining are summed to represent the bone development of different stages and to comprehensively assess the trend of osteogenesis at cranial defects of rats. The results show that the development of newly formed bone is significantly enhanced when either freshly frozen or freeze-dried Wharton's jelly products is implanted.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The terms “including” or “comprising” are usually used in the sense of including/including allowing for the presence of one or more features, ingredients or components. The terms “including” or “comprising” cover the terms “consisting of” or “consisting of”.

The term “individual” or “subject” includes a human and a non-human animal. For example, a companion animal (such as dogs, cats, etc.), a farm animal (such as cows, sheep, pigs, horses, etc.) or a laboratory animal (such as rats, and mice, guinea pigs, etc.).

As used herein, the term Wharton's Jelly refers to the Wharton's Jelly in the umbilical cord of mammals, which is also known as inter-laminar jelly, namely, the mucus connective tissue material found in the umbilical cord that consists of a large amount of extracellular matrix that forms a colloid with a three-dimensional architecture, which is rich in extracellular matrix, collagen, glycoproteins, and hyaluronic acid. The umbilical cord contains two umbilical arteries and one umbilical vein; and the connective tissue that surrounds the blood vessels is known as Wharton's Jelly.

A source suitable for preparing the product of the present invention is “Wharton's Jelly” from maternal umbilical cords, which may be processed by, for example, dissection, chopping, washing, enzymatic digestion, cryoprotection, freezing, drying, baking, or any combination thereof.

The term “critical-sized bone defect” as used herein refers to the destruction or removal of bone due to trauma, fracture, bacterial infection, or tumor metastasis and invasion. When the defect is more than twice the diameter of the long bone stem and does not heal naturally despite bone repair methods such as bone grafting, this phenomenon is called critical-sized bone defect.

The following examples are further provided to illustrate the present invention, and are intended to be illustrative only and not limiting. According to the present disclosure, it should be comprehended by those skilled in the art that there may be changes or modifications therein, and still obtain similar or like results without departing from the spirit and scope of the present invention.

### EXAMPLES

### Material and Methods

### 1.1 Preparation of Wharton's Jelly

Human umbilical cords were aseptically collected after delivery and kept in cold (4° C.) Hank's Balanced Salt Solution (HBSS; Gibco 14185-052). Wharton's jelly was isolated within 24 h after collection. All experimental instruments were autoclaved, disinfected in 75% ethanol, and flamed before using. In a laminar hood, umbilical cords were disinfected by soaking in 75% ethanol, placed in HBSS, and incised longitudinally. The blood vessels were detached and withdrawn to obtain Wharton's Jelly, which appeared as milky white interstitial tissues.

The isolated Wharton's jelly was subsequently processed using the following two methods:
Method (1). Frozen Wharton's Jelly (WJF): Freshly obtained Wharton's jelly was immediately frozen in the presence of a cryoprotective agent (10% DMSO) to obtain WJF, which was readily used after thawing.
Method (2). Decellularized Wharton's Jelly matrix (WJD): Freshly obtained Wharton's jelly was stored in a −80° C. refrigerator, then a freeze dryer was used to drain the moisture in tissues to obtain WJD, which were then sealed and stored in a dry box for use.

The WJF and WJD were used in the in vitro and animal experiments. In addition, the WJF and WJD were paraffin-embedded and subjected to histochemical staining to examine their histological structures.

### 1.2 Tissue Fibers Staining

The WJF and WJD were paraffin embedded. After deparaffinized and rehydrated, the tissue sections were stained using 0.1% Sirius red (Sigma 2610 Oct. 8) in picric acid. Subsequently, the tissue sections were washed by immersion in ddH₂O. Tissue sections were then dehydrated by immersing in a series of increasing concentrations of alcohol, followed by immersing in xylene. Finally, the section slides were mounted with mounting medium and examined via an optical microscope.

### 1.3 Tissue Glycoproteins Staining

After the WJF and WJD were deparaffinized and rehydrated, the tissue sections were stained with periodic acid-Schiff Stain (PAS stain) using the Periodic acid-Schiff Stain kit (Sigma 395B-1KT). The tissue sections were stained in periodic acid solution for 5 minutes, washed thrice in ddH₂O, 2 minutes for each time. And then, the tissue sections were stained in Schiff's reagent for 15 minutes, washed in running ddH₂O for 5 minutes. After staining, the interstitial glycoproteins are pinkish-purple. Next, the tissue slices were immersed in alcohol with increasing concentrations for dehydration, then immersed in xylene, and finally sealed for optical microscopic observation and photography.

### 1.4 In Vitro Culture of Rat Osteoblast (ROB)

One-day-old Sprague Dawley (SD) rats were sacrificed via anesthetic overdose, and in a laminar flow cabinet, the rats' calvarias were obtained using sterilized instruments and cut into cubes with a side length of 1 mm, followed by washing with sterile HBSS to remove red blood cells. The tissues were then reacted with 0.1% trypsin and 0.1% collagenase for 2 hours at 37° C. in an oven under shaking. Subsequently, Dulbecco's Modified Eagle Medium (DMEM) with 10% fetal bovine serum (FBS) was added to terminate the effect of trypsin, followed by centrifugation at 1500 rpm for 5 minutes. The cells attached to the bottom were re-suspended in DMEM with 10% FBS. Cells were seeded in a 10-cm culture dish, and the culture medium was changed every 4 days. After 10 to 14 days, a full confluence could be reached, and cells were then passaged. Osteoblasts at the second passage were used in this example.

### 1.5 Grouping of In Vitro Culture of Osteoblast

ROB cultured in vitro were divided into 5 groups as follows: (1) osteoblast group: 2×10⁴ ROB/well were cultured alone in a 24-well plate; (2) osteoblast/WJD group: 2×10⁴ ROB/well were seeded in the lower chamber of 24-well plate, and WJD was added to the top chamber of transwell; (3) osteoblast+WJD group: 2×10⁴ ROB/well were seeded in 24-well plate, and cultured with WJD; (4) osteoblast/WJF group: 2×10⁴ ROB/well were seeded in the lower chamber of 24-well plate, and WJF was added to the top chamber of transwell; (5) osteoblast+WJF group: 2×10⁴ ROB/well were seeded in 24-well plate, and cultured with freshly WJF. Culture medium (DMEM with 10% FBS) was changed every 3 days. Alkaline Phosphatase Cell Stain were assessed after 14 days of culturing.

### 1.6 Reverse Transcription-Polymerase Chain Reaction (RT-PCR)

Cells treated with 0.05% trypsin and fresh calvarial tissue were then reacted with trizol (Sigma T9424) to extract total RNA by centrifugation at 12000 g for 15 minutes at 4° C. The supernatant was mixed with equal volume of isopropyl alcohol and then centrifuged at 12000 g for 10 minutes at 4° C. The RNA was washed with alcohol, and then redissolved with DEPC water. The RNA concentration was measured before use. The purified RNA was then transcribed to complementary DNA (cDNA) via reverse transcription (RT). The cDNA was subjected to polymerase chain reaction (PCR) with primers and master mix. The following primers were used:

### Rat-Runt-Related Transcription Factor 2 (Rat-RUNX2) (212 bp)

Forward:
5′-GCTTCTCCAACCCACGAATG-3′
Reverse:
5′-GAACTGATAGGACGCTGACGA-3′

### Rat-GAPDH (160 bp)

Forward:
5′-CTCTACCCACGGCAAGTTCAAC-3′
Reverse:
5′-GGTGAAGACGCCAGTAGACTCCA-3′

The final PCR products were analyzed using 2% agarose gel by DNA electrophoresis and photographed with a UV transilluminator.

### 1.7 Alkaline Phosphatase Cell Staining

Cells were washed by saline solution, fixed using 2.5% glutardialdehyde for 15 minutes, dissolved with 5-Bromo-4-Chloro-3-Indolyl Phosphate/Nitro Blue Tetrazolium (BCIP/NBT, Sigma B5655) lozenges in 10 mL secondary water, and then added to a 24-well plate. After reaction at 37° C. in an oven in the dark for 1 hour, 10% SDS in hydrogen chloride (HCl) was added (Ocarino et al., 2008) to react at 37° C. in the oven in the dark for 12 hours. The absorbance of supernatant at 595 nm was measured to quantify alkaline phosphatases of each group. A higher absorbance indicates a higher level of alkaline phosphatase, representing more cells differentiating into osteoblasts.

### 1.8 Alizarin Red S Staining

ROBs of osteoblast group, osteoblast/WJD group, and osteoblast/WJF group were cultured with osteogenic medium (R&D CCM007) for 21 days (the medium was changed every 3 days). Cells were washed with saline, fixed using 2.5% glutardialdehyde for 15 minutes and then stained with 2% alizarin red S (referred to as ARS, Sigma A5533) for 1 hour in a 37° C. oven in the dark. Subsequently, 10% cetylpyridinium chloride (referred to as CPC, Sigma C-0732) (Qiao et al., 2016) was added to react for 1 hour in a 37° C. oven in the dark. The absorbance of supernatant at 540 nm was measured and quantified the red area of each group. A higher absorbance indicates a higher level of calcium precipitation.

### 1.9 Experimental Animals

The experimental animals used in this experiment were eight-week-old male Sprague Dawley (SD) rats weighed 250 to 280 g. The feeding method used a 45×24×20 cm transparent PC cage, 12 hours of light per day (7:30 am to 7:30 μm), constant temperature air conditioning (22±2° C.) for controlling ambient temperature and adequate supply of feed and drinking water (Laboratory Animal Diets MF18), and the litter was changed once a week.

### 1.10 Establishment of Critical-Sized Cranial Bone Defect Model in Rats

SD rats were anesthetized via intraperitoneal injection of Zoletil 50 and xylazine hydrochloride (Sigma 23076359), the rat's head was fixed in a stereotaxic apparatus and the hair was shaved cleanly with a razor. The rat's skin was then disinfected with iodine solution-dipped cotton ball and incised with a scalpel, the subcutaneous tissues were pushed away using a surgical scissor, and the periosteum was removed. Subsequently, a surgical trephine bur (8 mm in diameter) was applied to drill the calvaria, and the calvaria was detached from the underlying dura to serve as a critical-sized bone defect animal model. Finally, the subcutaneous tissues and skin were sutured.

### 1.11 Grouping of Experimental Animals

SD rats were grouped into 3 groups:
1. Injury+Saline group: no treatment but saline was given following calvarial bone defect.
2. Injury+WJD group: dissolved WJD in saline was administered to the defected region immediately after calvarial bone defect.
3. Injury+WJF group: dissolved WJF in saline was administered to the defected region immediately after calvarial bone defect.

### 1.12 Micro-Computerized Tomography Scan (Micro-CT)

Micro-CT images were processed with AMIDE software (SOURCEFORGE) to reconstruct 3-dimentional radiographic images. The Small Animal Computed Tomography System (U-CTUHR, MI Labs) was applied in the present study to scan live rats' calvarias on Day 1 after surgery and monthly at Months 1, 2, 3, 4, and 5 to follow-up the healing levels of calvarial bone defect.

### 1.13 Sacrifice, Perfusion Fixation, Decalcification, and Paraffin Sections of Experimental Animals

SD rats were anesthetized via intraperitoneal injection of Zoletil 50 and xylazine hydrochloride, and fixed via perfusion of 4% paraformaldehyde and 7.5% picric acid in 0.01M PBS. The calvarias were obtained and placed in fixative solution for 24 hours. For decalcification, calvarias were then immersed in 20% EDTA in 9% NaOH solution for 9 days, with the solution changed twice. The decalcified calvarias tissues were washed in running water for 8 hours, followed by dehydration with increasing concentrations of alcohol solutions, and paraffin-embedded with xylene and pure paraffin. Subsequently, the samples were horizontally sectioned into 7-μm thick tissue slices with a paraffin microtome.

### 1.14 Hematoxylin & Eosin Staining (H&E Staining)

Tissue sections were deparaffinized, rehydrated, stained with hematoxylin (Muto Pure Chemicals; No. 3008-1) for 5 minutes, and then washed in running water for 30 minutes. After being placed in 70% alcohol for 3 minutes, the sections were placed in a ventilated environment for the remaining alcohol to evaporate. Subsequently, the tissue sections were stained with eosin (Muto Pure Chemicals; No. 3008-2) for 1 minute, then washed in running water for 15 minutes, dehydrated with increasing concentrations of alcohol solutions, and treated with xylene twice. Finally, the slides were mounted with mounting medium for examination and photography via an optical microscope. The slice with the largest area of calvarial tissue was obtained, and the circumference of the boundaries between the old and newly formed bone were marked for quantification of the original area of bone defect. Subsequently, the newly formed bone within the bone defect region was quantified and expressed in percentage.

### 1.15 Alkaline Phosphate Staining (ALP Staining)

Calvarial tissue sections were deparaffinized, rehydrated, and reacted with Alkaline Phosphatase kit (Sigma 85L2-1KT) for 1 hour in a 37° C. oven, followed by immersion in ddH₂O for 2 minutes. The slides were mounted with mounting medium for examination, photography and quantification via an optical microscope. Stained sections could be observed a cell stained in purple, indicating that the sections contained ALP, i.e., the cell was a mature osteoblast. The slice with the largest area of calvarial tissue was obtained, and the circumference of the boundaries between the old and newly formed bone were marked for quantification of the original area of bone defect. Subsequently, the newly formed bone within the bone defect region was quantified and expressed in percentage as an area proportion of the osteoblasts.

### 1.16 Anti-Osteocalcin Antibody Immunohistochemistry Staining

Following deparaffinization of cranial tissue sections, primary mouse anti-osteocalcin antibody (Abcam ab13740, 1:200) was added to react at 4° C. for 16 to 18 hours. Subsequently, secondary goat anti-mouse-IgG-conjugated biotin (Millipore AP124B, 1:250) was added to react at room temperature for 1 hour, followed by reaction with VECTASTAIN® ABC-conjugated HRP Kit (Vector Laboratoris PK-4000) at room temperature for 1 hour. The sections were then washed thrice with 0.01M PBS for 5 minutes. Finally, DAB (5 mg DAB, 35% H₂O₂ 3.5 μL in 10 mL Tris-HCl, pH 7.4) was added for color development. After the staining was completed, the sections were air dried at room temperature. The sections were dehydrated by immersion in alcohol with increasing concentrations, followed by immersion in xylene. Finally, the slides were sealed with sealing glue and observed and photographed under an optical microscope.

All sections of calvarial tissue were taken and the percentage of osteocalcin (marked in dark brown) within the defect area was quantified to indicate the activity of ossification.

### 1.17 Statistical Analysis

All experimental data were expressed as mean±standard deviation. The comparison between the mean values was performed by one-way ANOVA or two-way ANOVA, followed by Fisher's least significant difference (LSD) test for multiple comparisons. Experimental data were all based on p<0.05 as the minimum starting standard for significant differences.

### 2. Results

### 2.1 Histological and Morphological Analysis of WJD and WJF

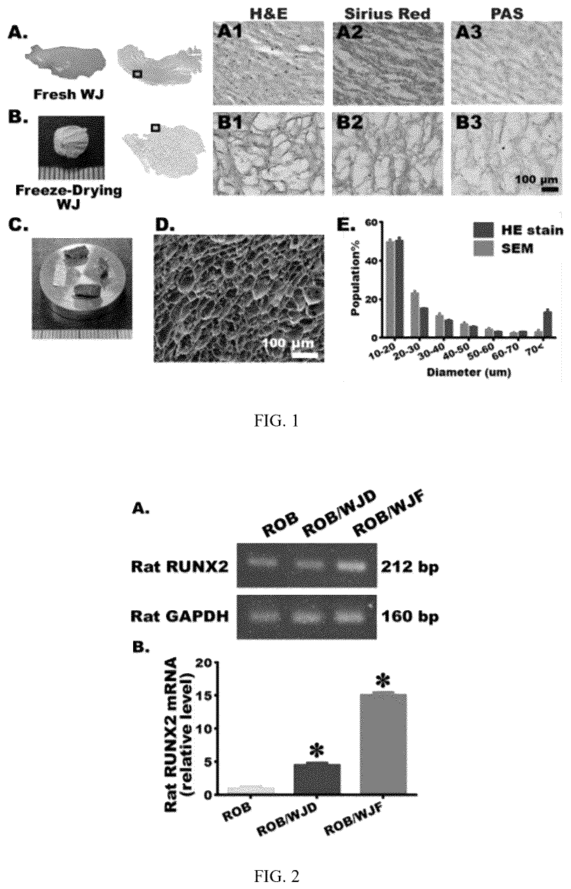

Freshly frozen Wharton's jelly (WJF) products (FIG. 1A) and freeze-dried Wharton's jelly (WJD) products (FIG. 1B) removed from the umbilical cord were embedded in paraffin for serial tissue sectioning. With H&E staining, the cellular and tissue structures of Wharton's Jelly were observed. Results showed that there were a large number of nuclei in WJF, and the structure was also dense and neatly arranged (FIG. 1A1). As to the lyophilized Wharton's Jelly, no nuclei could be observed, the structure was turned from dense to loose, and holes of various sizes were showed (FIG. 1B1). The main components of Wharton's Jelly were the collagens and glycoproteins, which were stained with Sirius red and PAS respectively to observe the distribution of the collagens and glycoproteins. Results showed that the collagens and glycoproteins in Wharton's Jelly were not destroyed by lyophilization, and still served as a supporting structure for Wharton's Jelly (FIG. 1A2-A3, B2-B3). The cell morphology was observed via a scanning electron microscope (FIG. 1C). Scanning images showed that WJD sections exhibited densely distributed pores (FIG. 1D). Multiple fields of view were randomly selected, holes were framed for size quantification, and the distribution of average hole diameters was calculated. Analysis results showed that WJD, whether paraffin-embedded for sectioning and quantified by H&E staining, or analyzed via a scanning electron microscope, exhibited the multiple holes structure of various sizes; and the hole diameter distribution mostly falls within the range of 10 to 70 microns (FIG. 1E).

### 2.2 Co-Culture of Rat Osteoblasts with Wharton's Jelly Products Enhances the Expression of Bone Differentiation Genes in Osteoblasts

In vitro cultures of rat osteoblasts (ROB), either cultured individually, or co-cultured with WJD or WJF for 14 days, were subjected to trypsin treatment and then the lower layer of cells were taken for RNA extraction. RT-PCR was performed using rat runt-related transcription factor 2 (RUNX2) to discuss the bone differentiation of ROB. Results showed that GAPDH was detected in the ROB group, ROB/WJD group, or ROB/WJF group; and RUNX2 was also detected in all groups (FIG. 2A). Further, differences in the expression level of gene between groups were analyzed using a real-time quantitative polymerase chain reaction apparatus. Quantitative results showed that the expression level of rat RUNX2 on osteoblast in ROB/WJD group or ROB/WJF group was significantly increased, compared with the ROB group (FIG. 2B).

### 2.3 Co-Culture of Rat Osteoblasts with Wharton's Jelly Products Enhances the Expression of Alkaline Phosphatase in Osteoblasts

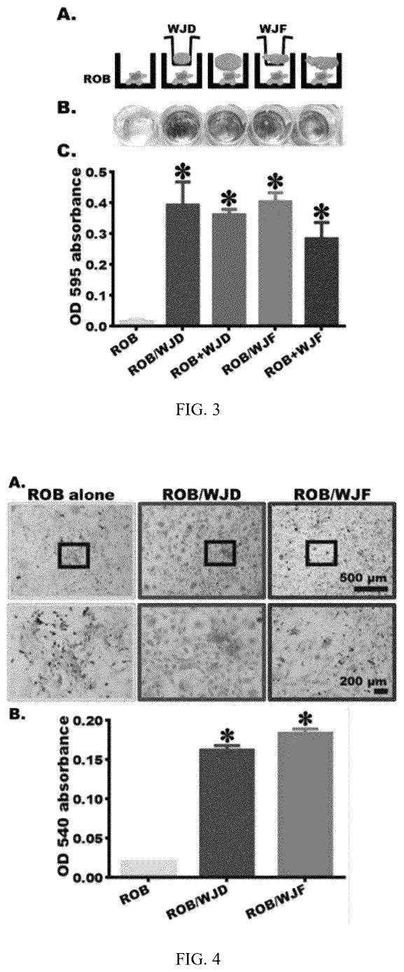

ROB were cultured in vitro individually, or co-cultured non-directly or directly in vitro with WJD and WJF respectively, and then treated with DMEM medium with 10% FBS for 14 days, then the cells were stained with BICP/NBT, and absorbances were measured by OD595 to observe the expression level of alkaline phosphatase in ROB (FIG. 3A, FIG. 3B). The results showed that the intracellular expression of alkaline phosphatase in ROB co-cultured non-directly or directly with WJD or WJF was significantly increased, compared with that of osteoblasts cultured individually (p<0.05, FIG. 3C).

### 2.4 Co-Culture of Rat Osteoblasts with Wharton's Jelly Products Increases the Level of Calcium Deposition

ROB were cultured in vitro individually, or co-cultured non-directly or directly in vitro with WJD and WJF respectively, and then treated with bone differentiation medium for 21 days. The cells were then stained with alizarin red S, and the manifestation of calcium deposition in ROB was marked in dark red color. With gradually zooming in from a low-magnification image, the osteoblasts in the ROB group were more sparsely clustered, and there was only a low level of calcium deposition in the field of view. In addition to clustered osteoblasts, the ROB/WJD group also had osteoblasts growing in concentric circles, and there was a high level of calcium deposition in the surrounding area. Mature osteoblasts with tubules could be found in the ROB/WJF group, and clusters of osteoblasts were surrounding nearby. There were also a large number of calcium crystals distributed in the field of view (FIG. 4A). Subsequently, the calcium crystals marked in dark red were dissolved, and the concentration of calcium ions was analyzed by measuring the absorbance with OD540. Results showed that the concentration of calcium ions in the ROB/WJD group or the ROB/WJF group was significantly increased, compared with the ROB group (p<0.05, FIG. 4B). It was hypothesized from the results of alizarin red S staining that non-directly co-culturing with WJD or WJF could promote the differentiation of osteoblasts and enhance the manifestation of calcium deposition.

### 2.5 Quantification of Bone Regeneration in Critical-Sized Calvarial Defect Via Micro-Computed Tomography Scan Shows that Wharton's Jelly Products Effectively Increase the Bone Production

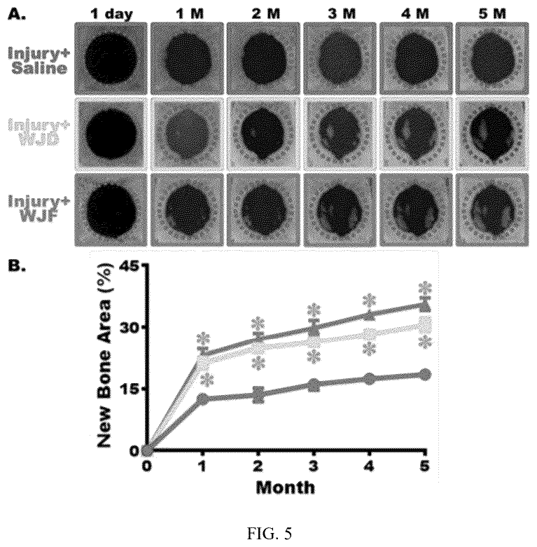

Calvarial bone healing was observed via micro-computed tomography scan (micro-CT) at the first, second, third, fourth, and fifth months after calvarial defect surgery in rats. The newly formed bone grew inwardly along the border of the bone defect in a small amount in the Injury+Saline group In addition to the inwardly growing new bone, sporadic new bone was also found in the defect area in the Injury+WJD group or Injury+WJF group (FIG. 5A). The percentage of new bone area within the calvarial defect area was quantified. Results showed that the Injury+Saline group had 12.5±3.4% of newly formed bone at the first month after surgery, the calvarial healing slowed down as time passed after surgery, and only 18.4±1.4% of the calvarial bone recovered at the fifth month after surgery. In the Injury+WJD and Injury+WJF groups, the percentages of newly formed bone were 21.2±3.9% and 23.0±4.1% respectively at the first month after surgery, which were significantly increased (p<0.05) compared with the Injury+Saline group, and the increasing was maintained until the fifth month, with the percentage of newly formed bone within the calvarial defect reaching 30.6±3.6%, and 35.6±3.7% respectively (p<0.05, FIG. 5B). The calvarial bone healing capacity of rats being operated for transcranial bone defects was only maintained in the range of 12% to 18% without any treatment over a period of five months, as macroscopically observed via micro-CT. Administration of the WJD or WJF product could promote osteogenesis in rats with calvarial bone defects.

### 2.6 H&E Staining Shows that Wharton's Jelly Products Promote the Bone Regeneration at Critical-Sized Calvarial Defects of Rats

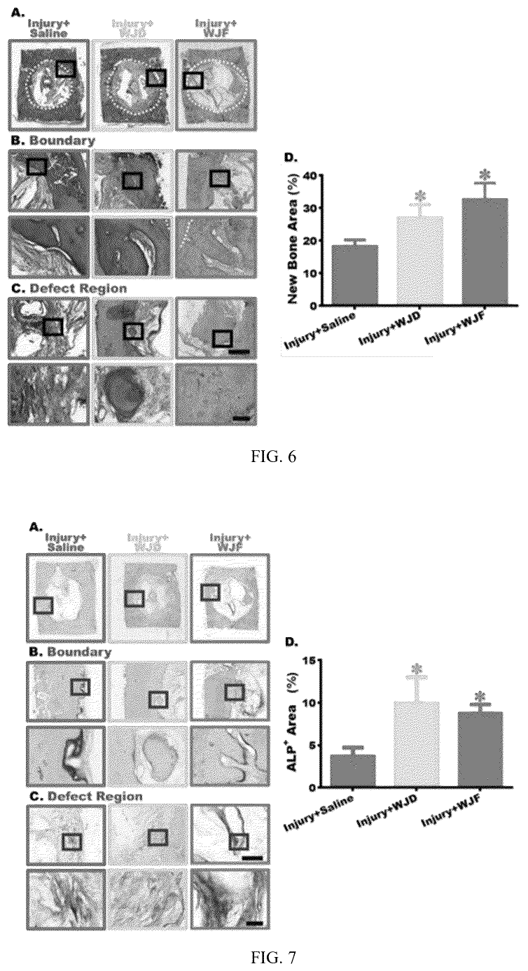

A low magnification panoramic image of the H&E staining results (FIG. 6A) was used to gradually magnify the bony performance from low magnification at the border of bone defect (FIG. 6B) and the internal area of bone defect (FIG. 6C). Results showed that there was a small amount of newly formed bone and dense connective tissue at the border of the defect in the Injury+Saline group; and there was only connective tissue within the bone defect. In addition to the tendency to increase the amount of bone at the border of the bone defects, free and maturing new bone was also observed within the bone defect in the Injury+WJD and Injury+WJF groups. The percentage of new bone area within the bone defect was quantified by H&E staining at the fifth month after surgery. There was only 18.8±1.9% of newly formed bone within the bone defect in Injury+Saline group. In contrast, the percentage of new bone area within the bone defect reached 27.1±4.0% and 32.7±5.0% in the Injury+WJD and Injury+WJF groups, respectively, which was a significant increase compared with the Injury+Saline group (p<0.05, FIG. 6D). As microscopically observed by H&E staining, administration of the WJD or WJF product promoted bone neogenesis in rats with calvarial defects.

### 2.7 Osteoblast Marking Via Alkaline Phosphatase Staining Shows that Wharton's Jelly Products Increase the Active Performance in Critical-Sized Calvarial Defect of Rats

A low magnification panorama image of the ALP staining results (FIG. 7A) were showed to gradually magnify the mature osteoblast expression area from low magnification at the border of defect (FIG. 7B) and the internal area of bone defect (FIG. 7C). Results showed that a small number of mature osteoblasts in the Injury+Saline group exhibited ALP at the border and internal of the bone defect. The mature osteoblasts adhered to inwardly growing new bone at the border of the bone defect in both the Injury+WJD and Injury+WJF groups, while free new bone was surrounded by osteoblasts within the bone defect. The percentage of ALP⁺ area within the defect was quantified by ALP staining. ALP⁺ area was only about 3.7±2.1% in the Injury+Saline group. ALP⁺ area reached 10.0±5.9% and 8.8±2.3% in the Injury+WJD and Injury+WJF treatment groups, respectively, which showed an increasing trend compared with the Injury+Saline group (p<0.05, FIG. 7D). As microscopically observed by ALP staining, administration of the WJD or WJF product increased the activity of osteoblasts in rats with calvarial defects.

### 2.8 Newly Formed Bone Marking Via Anti-Osteocalcin Antibody Immunohistochemistry Staining Shows that Wharton's Jelly Products Promote the Performance of Bone Regeneration at Critical-Sized Calvarial Defect of Rats

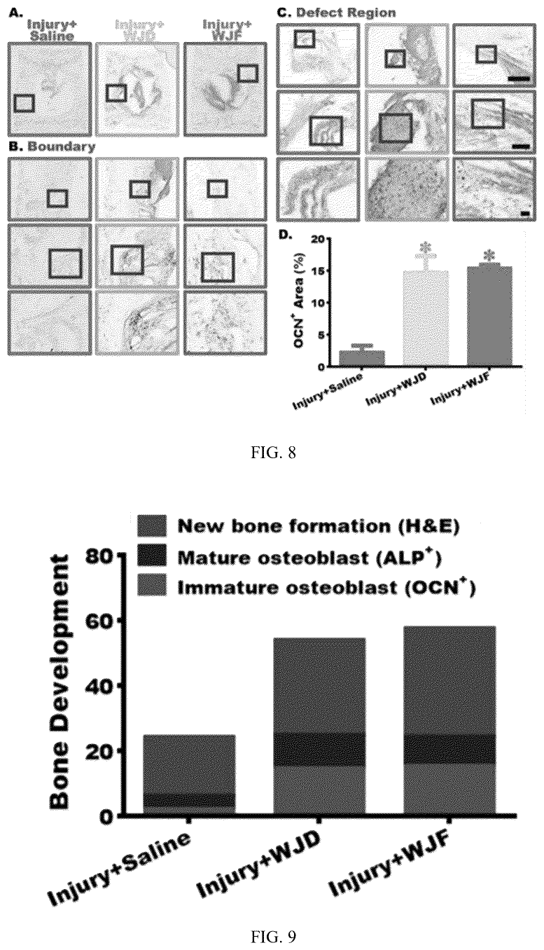

Osteocalcin is secreted by osteoblasts and released into bone tissue to form uncalcified bone during osteogenesis. A low magnification panoramic image of anti-osteocalcin immunostaining (FIG. 8A) results was showed to gradually magnify the expression level of osteocalcin-containing uncalcified bone at the border of the bone defect (FIG. 8B) and within the bone defect area (FIG. 8C). Results showed that in the Injury+Saline group there was no osteocalcin been observed at the border of the bone defect; and only a small amount of osteocalcin was released into the tissues within the bone defect. In the Injury+WJD and Injury+WJF individual groups, not only osteocalcin was found at the border of the bone defect and within the bone defect, but also clusters of osteoblasts were observed, which meant osteoblasts were undergoing an osteogenic reaction. The percentage of OCN⁺ area within the bone defect was quantified by OCN staining. There was only 2.4±1.6% of OCN⁺ area in the Injury+Saline group. In contrast, the percentage of OCN⁺ area reached 14.8±4.8%, and 15.5±1.5%, respectively in the Injury+WJD and Injury+WJF individual groups, which showed a significant increase compared with the Injury+Saline group (p<0.05, FIG. 8D). As microscopically observed by anti-osteocalcin staining, administration of the WJD or WJF product further promoted the expression of osteocalcin by osteoblasts, thereby promoting the regeneration of hard bone.

### 2.9 Wharton's Jelly Products could Obviously Promote the Development of Newly Formed Bone

Immature osteoblasts were marked with anti-osteocalcin immunostaining, mature osteoblasts were marked with ALP tissue staining, and mature bone were marked with H&E staining, respectively. Quantitative data from the three types of staining were summed to assess bone development in rats with calvarial defects. Results showed that the development of bone was approximately 25% in the Injury+Saline group, while approximately 52% and 57% in the Injury+WJD and Injury+WJF groups respectively indicated relatively high level of bone development (FIG. 9).

### 3. Conclusion

Wharton's Jelly products are transplanted into rats with calvarial defects. It is found that after treatment with either WJF or WJD, the new bone formation is observed by micro-CT, and the slice staining also proves that the signals presented in the images are mature bone, which also promotes the development of the host bone. According to the above experimental results, Wharton's Jelly products can be used as a natural medical material to promote bone regeneration in clinical medicine, so that patients who are suffering from bone defects can be provided with new treatment methods.

It is believed that a person with ordinary skill in the art to which the present invention belongs can utilize the present invention to the widest extent based on the description herein without further explanation. Therefore, the description and claims provided should be understood as being for illustrative purposes and not intended to limit the scope of the present invention in any way.

1.-7. (canceled)

8. A medical product for promoting bone regeneration, wherein the medical product is prepared by either of two methods below:
method (1): treating Wharton's Jelly taken from an umbilical cord with a cryoprotective agent, followed by cryopreservation, and thawing before use; or
method (2): directly lyophilizing the Wharton's Jelly taken from an umbilical cord, and storing in dry condition before use.

9. The medical product of claim 8, wherein the umbilical cord is from human or non-human mammals.

10. The medical product of claim 8, wherein the cryoprotective agent is a 10% dimethyl sulfoxide (DMSO) solution.

11. The medical product of claim 8, wherein the cryopreservation is performed in liquid nitrogen.

12. The medical product of claim 8, wherein the Wharton's Jelly under the cryopreservation contains cell bodies.

13. The medical product of claim 8, wherein the lyophilizing comprises taking a piece of fresh Wharton's Jelly from the umbilical cord, followed by direct cryopreservation at −80° C., and then draining moisture from tissues using a freeze dryer.

14. The medical product of claim 13, wherein the Wharton's Jelly contains no cell bodies.

15. The medical product of claim 8, which increases performance of alkaline phosphatase in the osteoblasts, increases level of expression of bone differentiation genes in the osteoblasts, promotes differentiation of the osteoblasts, enhances calcium deposition, effectively enhances production of bone, enables osteogenesis within a bone defect, enhances activity of the osteoblasts within the bone defect, or induces development of newly formed bone.

16. A use of the medical product of claim 8 in preparation of a medicament for promoting bone regeneration.

17. The use of claim 16, wherein the medical product increases performance of alkaline phosphatase in osteoblasts, increases level of expression of bone differentiation genes in the osteoblasts, promotes differentiation of the osteoblasts, enhances calcium deposition, effectively enhances production of bone, enables osteogenesis within a bone defect, enhances activity of the osteoblasts within the bone defect, or induces development of newly formed bone.

18. The medical product of claim 9, wherein the Wharton's Jelly under the cryopreservation contains cell bodies.

19. The medical product of claim 10, wherein the Wharton's Jelly under the cryopreservation contains cell bodies.

20. The medical product of claim 11, wherein the Wharton's Jelly under the cryopreservation contains cell bodies.
